(19) United States
(12) Patent Application Publication
(10) Pub. No.: US 2008/0281384 A1
(43) Pub. Date: Nov. 13, 2008

(54) Tanning system with video monitor

(76) Inventor: Bradley L. Tebo, Beverly, MA (US)

Correspondence Address: Paul J. Cook, 115 Pine Street, Manchester, MA, 01944, US

(21) Appl. No.: 11/801,757

(22) Filed: May 11, 2007

(51) Int. Cl. A61N 5/06

(52) U.S. Cl. 607/91

(57) ABSTRACT

A tanning bed or booth including means for generating video images connected to a monitor for displaying video images. The monitor is positioned within an opening through a cover, door or a housing for the bed or booth.

### FIELD OF THE INVENTION

This invention relates to a tanning system with a video monitor and a device for transmitting a video image to the video monitor.

### BACKGROUND OF THE INVENTION

At the present time horizontal or vertical tanning systems are provided for customer desirous of tanning their skin. Typically, tanning services provide individual booths or beds which accommodate a single customer. While in the tanning booth or bed, the customer is exposed to ultraviolet (UV) which causes the exposed skin to tan. In addition, it is normal business practice to schedule customers in a manner such that the customer has a short wait prior to entering the bed or booth.

Accordingly, it would be desirable to provide a tanning system wherein the waiting customers have their attention occupied prior to entering the tanning bed or booth. Such a system would reduce the tedium of the customer and provide information while waiting to enter the tanning bed or booth.

### SUMMARY OF THE INVENTION

A tanning system which can be horizontally positioned (tanning bed) or vertically positioned (tanning booth) is provided with a video monitor which is visible to a waiting customer through an opening in an outer wall of the bed or booth. The video monitor is connected to a device for transmitting a video image to the monitor such as a DVD player or the like. Optionally, speakers can be connected to the DVD or monitor in order to provide a sound message to the customer. The system is useful to provide the customer with visual information including a changeable decor, promotional information, instructional information, health information or the like.

### BRIEF DESCRIPTION OF THE DRAWINGS

FIG. 1 is a perspective view of a tanning booth of this invention.

FIG. 2 is the booth of FIG. 1 with its door open.

FIG. 3 is a front perspective view of a tanning bed of this invention.

FIG. 4 is a back perspective view of the bed of FIG. 3.

### DESCRIPTION OF SPECIFIC EMBODIMENTS

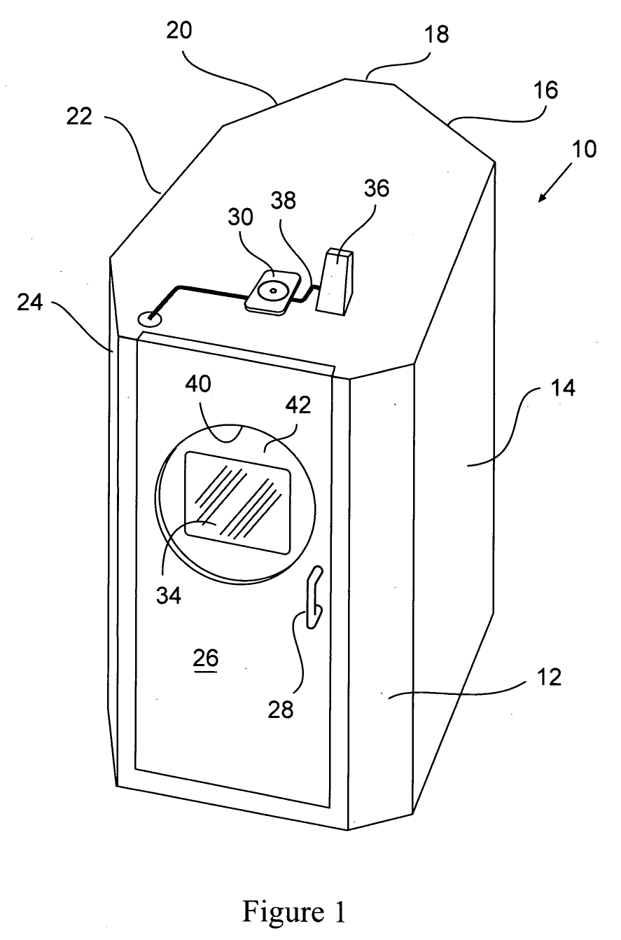

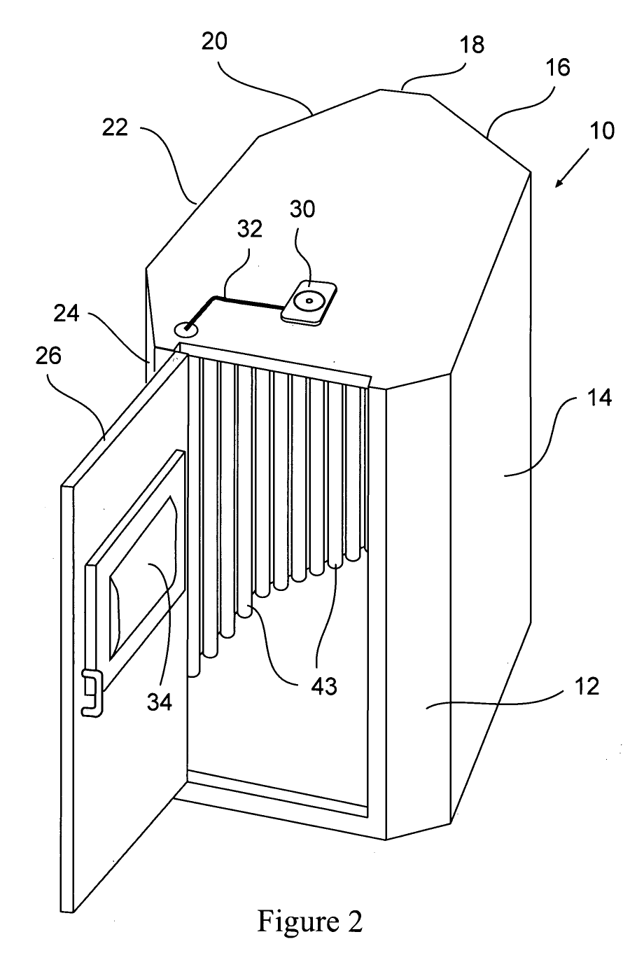

Referring to FIGS. 1 and 2, a tanning booth 10 includes vertical interconnected walls 12, 14, 16, 18, 22 and 24. A door 26 having a handle 28 is hung between walls 12 and 24 and functions to enclose the customer within the booth 10. A device for transmitting a video image such as a DVD player 30 is connected with connector 32 to a video monitor 34 in a conventional manner to effect a video image in monitor 34. An optional sound speaker 36 can be electrically connected to video image transmitter 30 through connector 38 to provide sound information to a customer. The mounter is positioned and supported in a stable manner within opening 40 so that it extends through the door 26 and which optionally can also enclose a transparent material 42 such as plastic or glass. The booth 10 includes tanning lamps 43 that provide a tan on the customer when activated. In use, the video transmitter 30 and monitor 34 are activated to produce a visual image in monitor 34. The speaker 36 can be optionally activated.

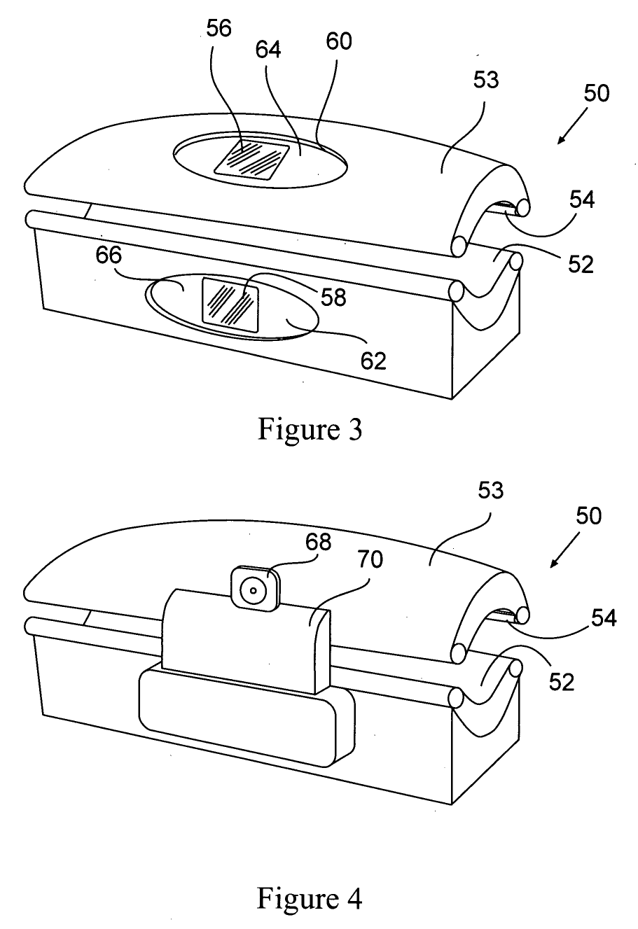

Referring to FIGS. 3 and 4, the tanning bed 50 includes a customer support 52 and a cover 53 to which are attached UV tanning lamps 54. A video monitor 56 and/or 58 are secured respectively to the cover 53 or the support 52. The cover 53 has an opening 60 therethrough to secure and support monitor 56. Support 52 has an opening 62 to secure and support monitor 58. Both monitors 56 and 58 can be viewed by a waiting customer through openings 60 and 62. Each opening 60 and 62 can enclose a transparent material 64 and 66 to permit viewing of the monitor 56 and/or 54 by a waiting customer. A device for transmitting a video image such as a DVD 18 is secured to the bed 50. The bed 50 includes a hinge 70 for opening or closing the cover 53.

1. A tanning housing for tanning the skin of a human being which comprises a housing having an interior containing lamps for generating ultraviolet light, a portion of said housing comprising a door or a cover that can be opened or closed, said housing having at least one opening therethrough to house and support a monitor for video images and means for connecting said monitor to a video image generating means in a manner to permit viewing of said image through said opening.

2. The tanning housing of claim 1 wherein one of said opening extends through said door or said cover.

3. The tanning housing of any one of claims 1 or 2 wherein said housing stands in a vertical position.

4. The tanning housing of any one of claims 1 or 2 wherein said housing stands in a horizontal position.

5. The tanning housing of any one of claims 1 or 2 wherein said video image generating means is connected with means for generating sound.

6. The tanning housing of any one of claim 3 wherein said video image generating means is connected with means for generating sound.

7. The tanning housing of any one of claim 4 wherein said video image generating means is connected with means for generating sound.
